(19) United States
(12) Patent Application Publication
(10) Pub. No.: US 2022/0062476 A1
(43) Pub. Date: Mar. 3, 2022

(54) SHOPPING CART DISINFECTION SYSTEM

(71) Applicant: UVC Ultra-Clean, LLC, Hewitt, TX (US)

(72) Inventors: Jefferson Blake West, Austin, TX (US); Corbett Benjamin Schoenfelt, Austin, TX (US); Gregory Lee Klepper, Elm Mott, TX (US)

(21) Appl. No.: 17/459,131

(22) Filed: Aug. 27, 2021

Related U.S. Application Data
(60) Provisional application No. 63/071,666, filed on Aug. 28, 2020.

(51) Int. Cl. A61L 2/10

(52) U.S. Cl. CPC A61L 2/10; A61L 2202/122; A61L 2202/11; A61L 2202/14

(57) ABSTRACT

This disclosure relates to a system for disinfecting shopping carts and a corresponding method. In an example system, a chamber and at least one UV LED configured to emit UV light into the chamber to disinfect shopping carts in the chamber.

### RELATED APPLICATIONS

This application claims the benefit of U.S. Provisional Application No. 63/071,666, filed Aug. 28, 2020, the entirety of which is herein incorporated by reference.

### TECHNICAL FIELD

This disclosure relates to a system for disinfecting shopping carts and a corresponding method.

### BACKGROUND

Shopping carts, which in some parts of the world are known by other names such as trolleys, are wheeled carts used by customers of a store, such as a supermarket, for the transport of merchandise from the shelves of the store to the checkout counter, and, in some cases, to a customer's vehicle. Shopping carts are typically supplied to the customer by the store. Shopping carts are often reused many times per day. Due to contact and exposure to previous users, it is known that shopping carts should be disinfected, or sanitized. Stores often provide antibacterial wipes to customers for use in disinfecting shopping carts.

### SUMMARY

A shopping cart disinfection system according to an exemplary aspect of the present disclosure includes, among other things, a chamber and at least one UV LED configured to emit UV light into the chamber to disinfect shopping carts in the chamber.

In a further embodiment, the system includes a push bar configured to be grasped by a user, and a paddle moveable in response to the push bar. The paddle projects into the chamber and is configured to apply a force onto shopping carts in the chamber to cause the shopping carts to move out of the chamber.

In a further embodiment, movement of the push bar results in greater movement of the paddle.

In a further embodiment, the push bar is operatively connected to the paddle by a first set of rollers on a same side of the chamber as the push bar and a second set of rollers on an opposite side of the chamber, and the second set of rollers exhibit a greater diameter than the first set of rollers.

In a further embodiment, the paddle is configured to rotate about an axis in only a single direction.

In a further embodiment, the paddle is biased to a neutral position and is configured such that rotation in a first direction from the neutral position is prevented and further such that rotation in a second direction from the neutral position opposite the first direction is permitted.

In a further embodiment, the paddle includes a ratchet assembly.

In a further embodiment, when shopping carts are in the chamber, the paddle is permitted to travel from a second end of the chamber to a first end of the chamber without causing movement of the shopping carts and is further permitted to travel from the first end of the chamber to the second end of the chamber while causing movement of the shopping carts.

In a further embodiment, the chamber is enclosed by a plurality of panels.

In a further embodiment, within the chamber, the system includes at least one reflective panel.

In a further embodiment, at least one of the plurality of panels is a first door arranged at an end of the chamber.

In a further embodiment, at least one other of the plurality of panels is a second door arranged at an end of the chamber opposite the first door.

In a further embodiment, the at least one UV LED includes a plurality of UV LEDs, and each of the plurality of UV LEDs are configured to emit UV light into the chamber.

In a further embodiment, the plurality of UV LEDs includes a first row of UV LEDs and a second row of UV LEDs, the first row of UV LEDs is on a first side of the chamber and is arranged at above a height corresponding to a handlebar of a shopping cart, and the second row of UV LEDs is on the first side of the chamber and is arranged at below the height.

In a further embodiment, the first and second rows of UV LEDs are inclined toward the height.

In a further embodiment, the first row of UV LEDs includes three light sources and the second row of UV LEDs includes three light sources.

In a further embodiment, the plurality of UV LEDs includes an upper row of UV LEDs adjacent a top of the chamber and configured to emit light downward toward the height.

In a further embodiment, the system includes a light configured to emit light of a color corresponding to a particular stage of a disinfection cycle.

A method according to an exemplary aspect of the present disclosure includes, among other things, disinfecting shopping carts arranged within a chamber by emitting UV light from at least one UV LED within the chamber.

In a further embodiment, the method includes removing the shopping carts from the chamber after the disinfecting step by applying a force to a push bar. The push bar causes a paddle projecting into the chamber to apply a force onto the shopping carts, and movement of the push bar results in greater movement of the paddle.

### BRIEF DESCRIPTION OF THE DRAWINGS

FIG. 1 is a perspective view of an example disinfection system from an end.

FIG. 2 is a perspective view of a frame of the example system.

FIG. 3 is a perspective view of a portion the example system from an end opposite that of FIG. 1.

FIG. 4 is a perspective view of the example system of FIG. 1, which includes doors at both ends of a chamber.

FIG. 5 is a perspective view of another example system including a door at only one end of a chamber.

FIG. 6 is a side view of the example system of FIG. 1 with a housing partially removed.

FIG. 7 is a cross-sectional view of the system illustrating an example arrangement of the chamber.

FIG. 8 is a perspective view of a portion of the example system from the same end as that of FIG. 1. In FIG. 8, a door adjacent the end is open.

FIG. 9 is a perspective view of a frame and other components of the system.

FIG. 9 includes two callouts illustrating additional detail of the system.

FIG. 10 is an exploded view of some components of the system.

### DETAILED DESCRIPTION

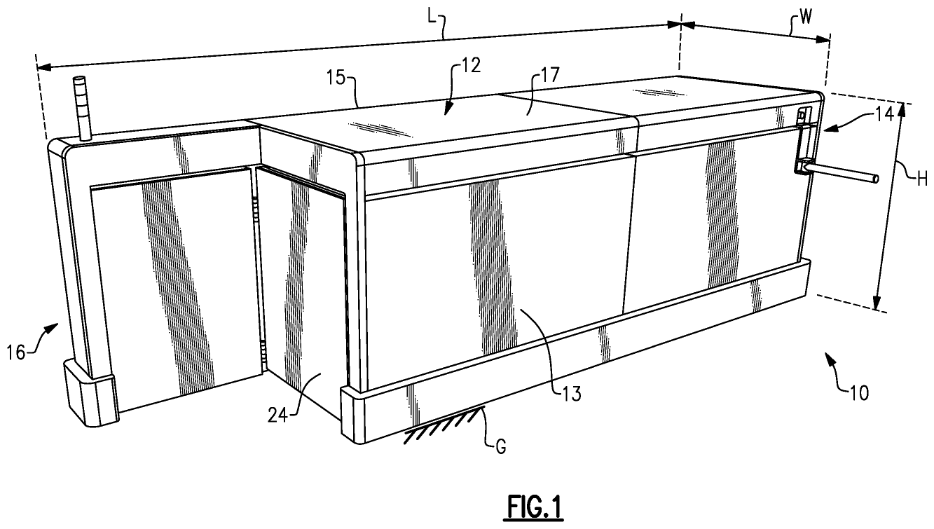

This disclosure relates to a system for disinfecting shopping carts. FIG. 1 is a perspective view of an example shopping cart disinfection system 10 (“system 10”). The system 10 includes a housing 12 extending along a length L between a first end 14 and a second end 16 opposite the first end 14. The housing 12 further exhibits a width W between opposed first and second sides 13, 15, and a height H extending from a ground, or floor, surface G to a top 17. In an example, the length L is about 195 inches (about 496 cm), the width W is about 53 inches (about 134 cm), and the height H is about 57 inches (about 145 cm).

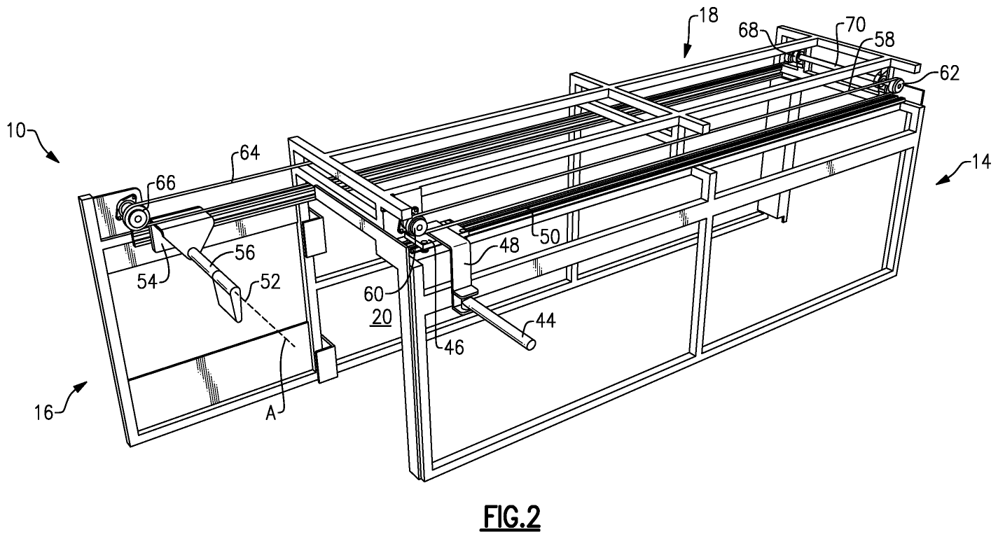

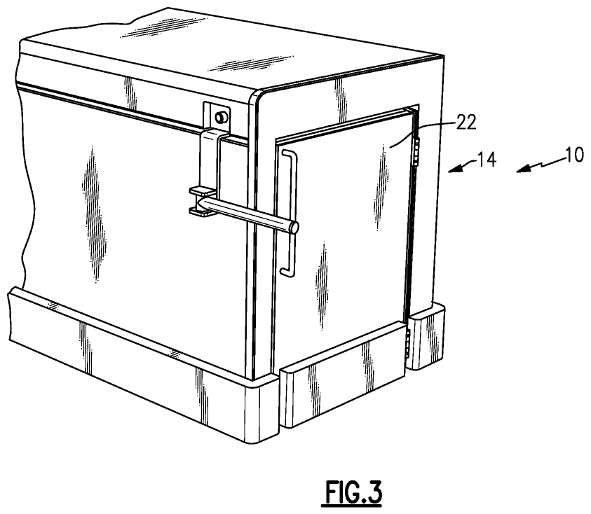

The housing 12 may include a plurality of panels, which may be metallic, mounted to a frame 18 (FIG. 2), which is provided by a weldment in one example. The panels enclose a chamber 20. The sides 13, 15 and top 17 may be provided by one or more panels, as generally shown in FIG. 10. In one example of this disclosure, two of the panels adjacent the first and second ends 14, 16 are doors that are hingedly mounted to the frame 18 and are configured to open and close to selectively provide access to the chamber 20 and seal the chamber 20, respectively. Specifically, the system 10 includes a first door 22 (FIG. 3) hingedly mounted adjacent the first end 14 and a second door 24 (FIG. 1) hingedly mounted adjacent the second end 16. The second door 24, in this example, is spaced-apart from the second end 16, in this example, in order to increase the ease of removing a row of shopping carts from the chamber 20.

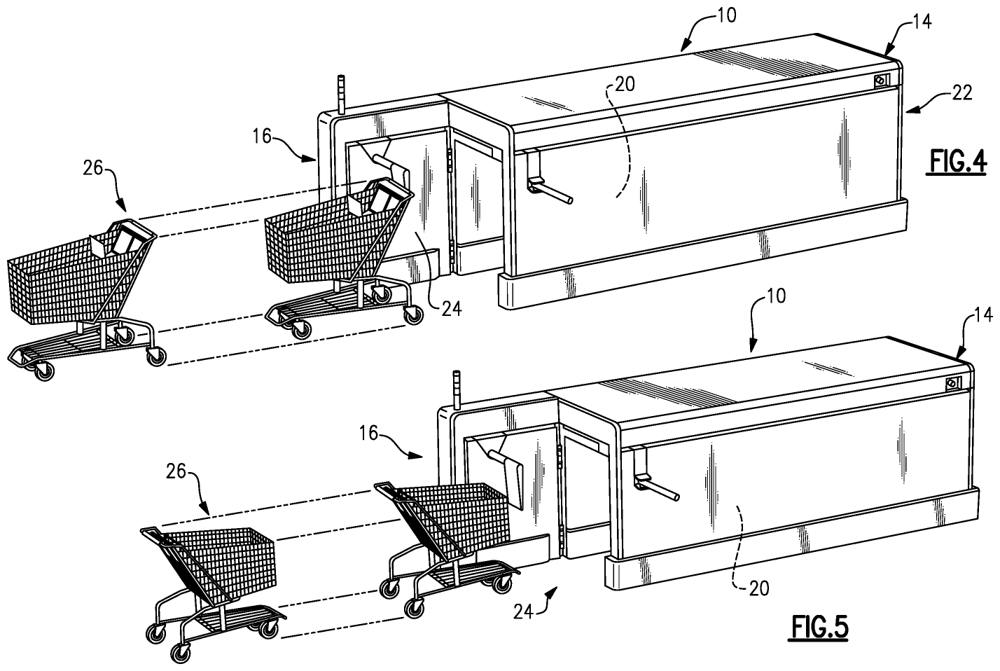

FIG. 4 illustrates the system 10, and in particular illustrates the system 10 with two doors, namely the first door 22 and the second door 24. When configured with two doors, one arranged at each of the first and second ends 14, 16, the system 10 is configured such that a row of shopping carts 26 (“carts 26”) can pass through the chamber 20 from the first end 14 to the second end 16. Note that in some figures a row of carts 26 is shown by two end carts 26 with dashed lines between those end carts 26. In this regard, the dashed lines represent additional carts. The chamber 20 provides a tunnel, which is configured to selectively open and close at both of its ends. In particular, in an example, unsanitized carts 26 enter the chamber 20 from the right-hand side of the first end 14 by traveling in the left-hand direction, and continued travel of the carts 26 results in the carts 26 exiting the chamber 20 via the second door 24. In FIG. 4, the second door 24 is in an open position.

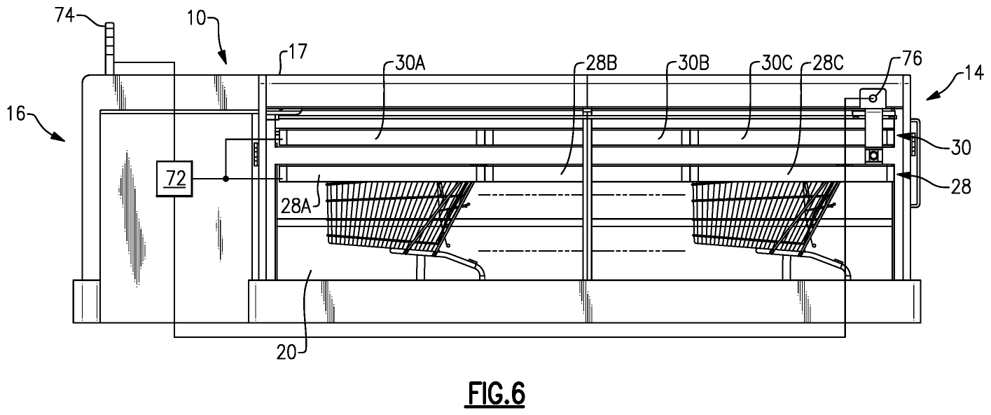

FIG. 5 illustrates an alternate arrangement of the system 10, and in particular illustrates the system 10 configured with only one door. In FIG. 5, the first door 22 is replaced with a fixed, non-hinged panel. Carts 26 enter the chamber 20 from the left-hand side of the second end 16 and exit the chamber 20 by moving backwards, in the left-hand direction, out the second door 24. The majority of this disclosure will refer to the embodiment of the system 10 with the first and second doors 22, 24, however it should be understood that this disclosure extends to embodiments with only one door. The embodiment with only one door may be particularly useful in situations where floor space is limited. The embodiment with two doors may be particularly useful by permitting relatively high throughput of carts.

The chamber 20 is sized and configured to fit a row of carts 26, such as about 8 carts nested together in a row. The chamber 20 could be sized to accommodate fewer or additional carts. The chamber 20 is sized and configured such that a row of carts 26 can fit in the chamber 20 when the first and second doors 22, 24 are closed. When inside the chamber 20, the system 10 is configured to disinfect the carts 26 by emitting ultraviolet (UV) light onto the carts 26. In this disclosure, a plurality of UV light sources are arranged in the chamber 20. Various aspects of the system 10 pertaining to the arrangement of the UV light sources, the manner in which carts 26 are fed through the chamber 20, and an example method of use, among other things, will now be described.

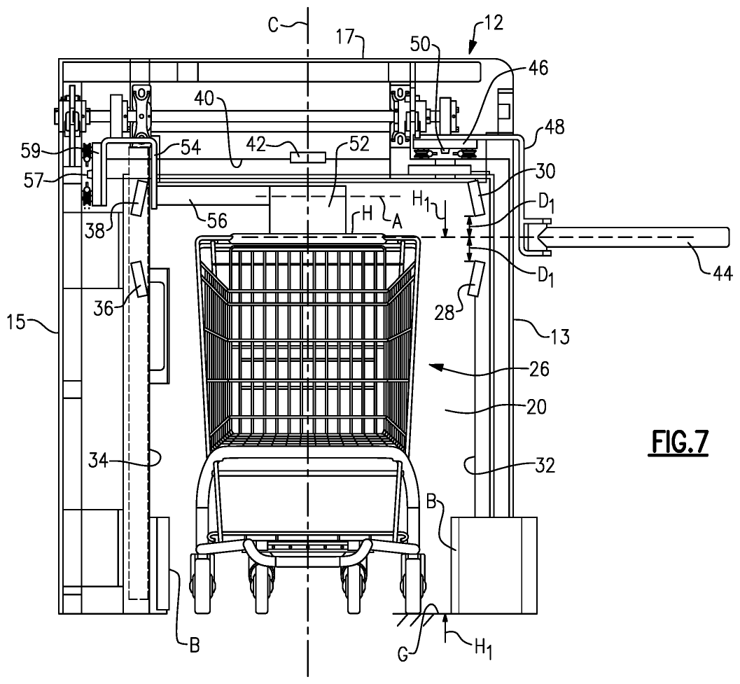

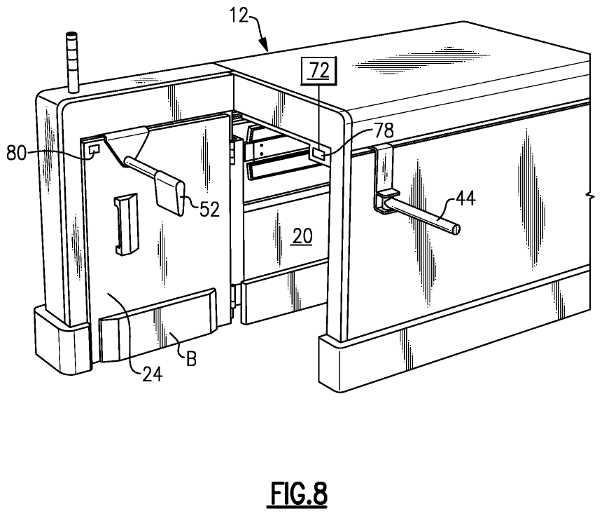

FIG. 6 is a side view of the system 10 with some panels removed so that the frame 18 and a partial arrangement of UV light sources are visible. FIG. 7 illustrates additional detail of the arrangement of UV light sources. Specifically, in this example, the arrangement of UV light sources includes a first row of UV light sources 28 extending substantially the entire length of the chamber 20 (i.e., substantially the entire length between the first and second doors 22, 24) and arranged beneath a height H₁ corresponding to a height of the handlebars H of the carts 26 above the ground surface G. In particular, the first row of UV light sources 28 is arranged beneath the height H₁ by a distance D₁. A second row of UV light sources 30 is arranged above the height H₁ by the distance D₁. The first and second rows of UV light sources 28, 30 are inclined toward the handlebars H such that light emitted from the first and second rows of UV light sources 28, 30 is focused on the handlebars H, which is an area of the carts 26 frequently contacted by customers.

The first and second rows of the UV light sources 28, 30 are mounted to a first side wall 32 of the chamber 20. On an opposite side of the chamber 20, the chamber 20 is bound by a second side wall 34. The first and second side walls 32, 34 could be provided by the sides 13, 15 or other walls spaced-inward of the sides 13, 15. In this example, there are third and fourth rows of UV light sources 36, 38 mounted to the second side wall 34 in the same manner as the first and second rows of UV light sources 28, 30. The third and fourth rows of UV light sources 36, 38 are essentially a reflection of the first and second rows of UV light sources 28, 30 about a centerline C of the chamber 20.

With reference to FIG. 6, the first row of UV light sources 28 includes three UV light sources 28A, 28B, 28C arranged at a common height and aligned end-to-end such that the first row of UV light sources 28 spans substantially the entire length of the chamber 20. The second row of UV lights sources 30 is likewise arranged, including three UV light sources 30A, 30B, 30C arranged at a common height and aligned end-to-end. The third and fourth rows of UV lights sources 36, 38 are likewise arranged. While three UV light sources are shown relative to each row, the first, second, third, and fourth rows of UV light sources 28, 30, 36, 38 could include one or more individual UV light sources. In an example, each of the individual light sources exhibits a height of about 9 cm. While two rows of UV light sources are on each of the side walls 32, 34, this disclosure extends to systems with one or more rows of UV light sources on each side wall.

Adjacent a top 40 of the chamber 20, the system 10 further includes an upper row of UV light sources 42. The top 40 could be provided by the top 17 or another panel spaced-apart below the top 17. The upper row of UV light sources 42 is configured to emit UV light in a direction toward the ground surface G, which, as a result, directs UV light toward the handles H of the carts 26 and the baskets of the carts 26. The upper row of UV light sources 42 is on the centerline C in this example. The upper row of UV light sources 42 may include a plurality, such as three, of individual UV light sources arranged end-to-end, similar to the first and second rows of UV light sources 28, 30. While only one upper row of UV light sources is shown, this disclosure extends to systems with one or more rows of upper UV light sources. Further, this disclosure extends to systems without an upper row.

Each of the light sources are UV light emitting diode (LED) light sources, in this example. The UV LED light sources are relatively robust, meaning they are able to withstand wear from normal use of the system 10. In particular, the UV LED light sources, as opposed to other types of UV light sources, are able to withstand repeated contact with the carts 26. The UV light sources are configured to emit UV-C light having a wavelength within a range of 245-290 nanometers (nm) in one example, and in a specific example at a wavelength of 254 nanometers (nm). UV-C light is a subtype of UV light especially suited for disinfection and is known to kill, break down, and/or inactivate microorganisms such as bacteria, germs, dust mites, mold, fungi, etc.

The chamber 20 includes a plurality of bumpers B arranged on an inside thereof for facilitating movement of the carts 26. Further, the second door 24 may also include a bumper B thereon, adjacent the ground surface G. The first door 22 may also include one or more bumpers.

Further, in order to ensure that the carts 26 receive an adequate dose of UV light, the chamber 20 may include one or more reflective panels configured to reflect the UV light emitted from the UV light sources such that the UV light is directed toward various portions of the carts 26. The chamber 20 may also include a reflective coating in addition to or as an alternative to reflective panels. That said, this disclosure extends to systems without a reflective coating or reflective panels.

Another aspect of the system 10 relates to retrieving the carts 26 from the chamber 20. With specific reference to FIGS. 2 and 7, the system 10 includes a push bar 44 (or a pull bar, depending on how a user chooses to apply force to the push bar 44) projecting outward, substantially parallel to the ground surface G, at the height H₁ above the ground surface G. The push bar 44 is connected to a guide 46 via an L-shaped bracket 48. The guide 46 is slidable along a rail 50, in response to corresponding forces applied to the push bar 44, along substantially the entire length of the chamber 20. The guide 46 may include rollers, or wheels, received in a corresponding track or slot of the rail 50.

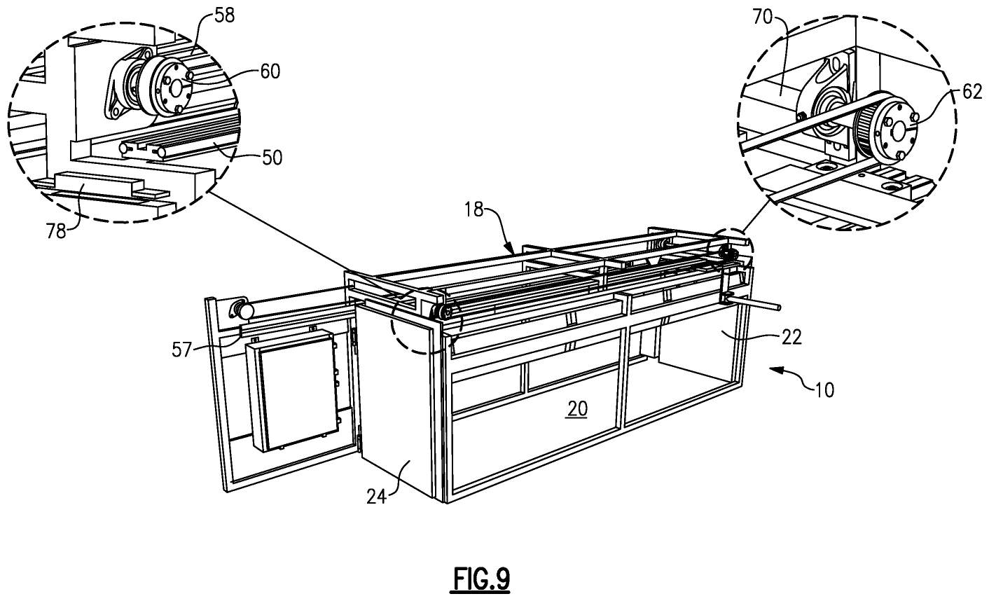

Movement of the push bar 44 results in corresponding movement of a paddle 52. The paddle 52 projects into the chamber 20 and is connected to a guide 59 via a first bar 54 and a second bar 56. The first bar 54 is substantially U-shaped, and in particular resembles an upside-down U (FIG. 7). The guide 59 is mounted to a left-hand leg (relative to FIG. 7) of the first bar 54, and is configured to travel along a rail 57. The first bar 54 is also shaped such that it can travel over a top of the second door 24 when the second door 24 is open (FIG. 8). Rail 57 is rotated 90° relative to rail 50, however this is not required. Both rails 50, 57 extend in a direction parallel to the length L. Rails 50, 57 have ends aligned with one another adjacent the first end 14, but the rail 57 is longer than the rail 50 and projects closer to the second end 16 than the rail 50, in this example.

The second bar 56 projects from a right-hand leg (relative to FIG. 7) of the first bar 54 into the chamber 20. The second bar 56 is arranged such that the paddle 52 is configured to contact the handlebars H of the carts 26. The paddle 52 could also be sized and configured to contact a front edge of the carts 26. In FIG. 7, the paddle 52 is mounted to the second bar 56 such that the paddle 52 projects downward from the second bar 56, toward the ground surface G, in a neutral position. The paddle 52 is arranged on the centerline C and is provided at the height H₁. Portions of the paddle 52 extend above and below the height H₁.

The paddle 52 is biased toward the neutral position of FIG. 2, in one example. Further, the paddle 52 is configured to rotate in the clockwise direction (relative to FIG. 2) about a central axis A of the second bar 56 from the neutral position in response to an applied force, such as a force applied by carts 26 being inserted into the chamber 20 from the first end 14. The paddle 52 is further configured such that, in response to a biasing member or gravity, the paddle 52 is biased to move back to the neutral position, and that rotation of the paddle 52 in the counter-clockwise direction (again, relative to the orientation of FIG. 2) beyond the neutral position is prevented. Thus, as the paddle 52 moves in a direction from the first end 14 toward the second end 16, the paddle 52 applies a force to the carts 26, via a handlebar H of an end one of the carts 26, and does not rotate about the axis A in the counter-clockwise direction. If carts 26 are present in the chamber 20, movement of the paddle 52 from the second end 16 to the first end 14 is not prevented because the paddle 52 is permitted to rotate in the counterclockwise direction. Once the paddle 52 reaches an end of the row of carts 26 (i.e., when the paddle 52 is adjacent the first end 14), the paddle 52 returns to the neutral position and can again apply a force to the carts 26. The paddle 52 is mounted to a free end of the second bar 56 by a ratchet assembly or another assembly that permits rotation in one direction but not another.

The push bar 44 is connected to the paddle 52, in this example, by way of a mechanical connection including belts and a shaft. In particular, the guide 46 is connected to a first belt 58, which is connected to first and second rollers 60, 62 mounted to the frame 18 at opposite ends of the chamber 20. The guide 59 is likewise connected to a second belt 64 mechanically connected to third and fourth rollers 66, 68 mounted to the frame 18 adjacent the first and second ends 14, 16. The rollers 60, 62, 66, 68 engage the respective belts 58, 64 via teeth that fit into corresponding teeth of the rollers 60, 62, 66, 68, in an example. The second and fourth rollers 62, 68, which are arranged adjacent the first end 14, are connected via a shaft 70.

The above-discussed mechanical connection is such that movement of the push bar 44 translates into movement of the paddle 52. In a particular aspect of this disclosure, movement of the push bar 44 translates into a greater movement of the paddle 52. Specifically, 1 unit of movement of the push bar 44 translates into about 1.3 units of movement of the paddle 52. As shown in FIG. 2, when the push bar 44 is adjacent a second end 16 of the system 10, the paddle 52 is not aligned with the push bar 44. Rather, the paddle 52 is closer to the second end 16 than the push bar 44. In this way, when the push bar 44 is pushed toward the second end 16, the paddle 52 travels out of the chamber 20, which increases the ease of retrieving the carts 26. In this regard, the end of the chamber 20 is spaced-apart from the second end 16 such that the paddle 52 can travel beyond the chamber 20 and remain within the housing 12. When the push bar 44 is at an opposite end, namely adjacent the first end 14, the push bar 44 and the paddle 52 are substantially aligned. This relationship between the travel of the push bar 44 and the paddle 52 is achieved by relative sizing of the rollers 60, 62, 66, 68. Namely, the rollers 66, 68 are of a greater diameter than the rollers 60, 62. The relationship could be achieved using other known mechanical techniques, such as gearing.

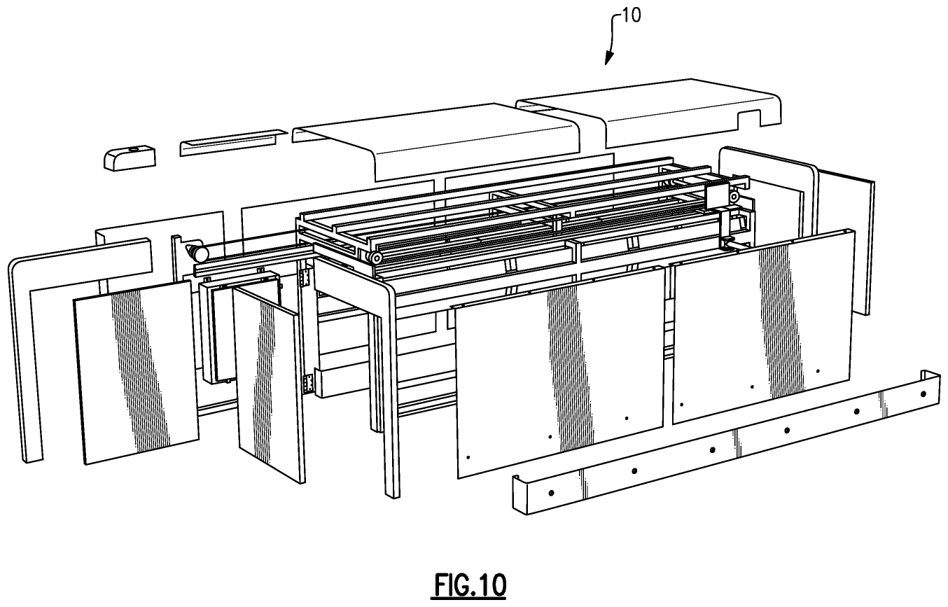

The system 10 includes a number of electrical components. In particular, the system 10 includes a controller 72 (which may be called a “control unit”) electrically connected to one or more additional electrical components of the system 10. The controller 72 is programmed with executable instructions for interfacing with and operating the various components of the system 10, including but not limited to those shown in the figures and discussed herein. It should also be understood that the controller 72 may include a combination of hardware and software, and specifically may include a processing unit and non-transitory memory for executing the various control strategies and modes of the system 10. The controller 72 and other electrical components of the system 10 may be mounted in a NEMA enclosure, such as that shown in FIG. 9, adjacent the second end 16 and the side 15.

In this example, the controller 72 is electrically connected to a light 74 projecting upward from a top 17 of the housing 12. The light 74 is configured to illuminate a plurality of different colors, and in particular includes three distinct lights, namely red, yellow, and green lights. The controller 72 is configured to cause the light 74 to illuminate a particular color to indicate a status of the system 10 to a user.

The controller 72 is also electrically connected to each of the UV light sources and is configured to selectively activate them (i.e., turn them “on”) and deactivate them (i.e., turn them “off”). The controller 72 is additionally electrically connected to locks for the first and second doors 22, 24 and a button 76 which, when pressed, is indicative of a user's desire to have the controller 72 lock the doors 22, 24 and begin a disinfection, or sanitization, cycle. An example magnetic lock 78 is illustrated in FIG. 8 relative to a second end of the chamber 20. The example magnetic lock 78 is an electromagnet which, when activated in response to instructions from the controller 72, is configured to attract a permanent magnet 80 arranged in the second door 24 to hold the second door 24 in the closed position. The first door 22 includes a similar magnetic locking arrangement, which is configured to selectively hold the first door 22 in the closed position in response to instructions from the controller 72.

An example method of use will now be described. In the example method, the chamber 20 begins as empty (i.e., not containing carts) and the first and second doors 22, 24 are in the open position. A user (i.e., a worker at a store) pushes a row of carts 26 into the chamber 20 via the first door 22. Either before or after pushing the row of carts 26 into the chamber 20, the user moves the push bar 44 to the first end 14, such as in the position of FIG. 1, such that the paddle 52 contacts a handlebar H of a cart closest the first door 22. The user then closes the first and second doors 22, 24. The light 74 is illuminated a green color indicating a “ready” state, such that a user understands the user can press the button 76 to activate a disinfection cycle. The user then presses the button 76 and, in response, the controller 72 activates the UV light sources for a period of time. During this period of time, which may be 30 seconds, the light 74 is illuminated a red color, indicating to the user that the disinfection cycle is active and the UV light sources are activated and emitting UV light within the chamber 20. Further, during this period of time, the controller 72 activates the magnetic locks, holding the doors 22, 24 in the closed position. After the period of time, the light 74 illuminates a green color again, indicating it is safe again to open the first and/or second doors 22, 24. The user opens the second door 24 and pushes the push bar 44 to the second end 16. The paddle 52 applies a force to the rearward-most of the carts 26 and, as the paddle 52 travels out of the chamber (as in FIG. 4) the carts 26 may be grasped by the user and moved away from the system 10 to a location where the carts 26 can be used by customers. The user may load additional carts into the chamber 20 and run the disinfection cycle again.

With one or more of the doors 22, 24 open, the light 74 may emit a yellow light indicating that the UV light sources are not activated but that one or more of the doors 22, 24 is open such that the button 76 cannot be pressed and/or that pressing the button 76 will not activate the disinfection cycle. If there is an issue with the system 10, the light 74 may flash a yellow color.

Directional terms such as “top,” “bottom,” “upward,” “downward,” etc., are used herein for purposes of explanation and with reference to the orientation of components illustrated in the drawings. Such directional terms should not be considered limiting. Further, it should be understood that terms such as “generally,” “substantially,” and “about” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples. In addition, the various figures accompanying this disclosure are not necessarily to scale, and some features may be exaggerated or minimized to show certain details of a particular component or arrangement.

One of ordinary skill in this art would understand that the above-described embodiments are exemplary and non-limiting. That is, modifications of this disclosure would come within the scope of the claims. Accordingly, the following claims should be studied to determine their true scope and content.

1. A shopping cart disinfection system, comprising:
a chamber; and
at least one UV LED configured to emit UV light into the chamber to disinfect shopping carts in the chamber.

2. The system as recited in claim 1, further comprising:
a push bar configured to be grasped by a user; and
a paddle moveable in response to movement of the push bar and projecting into the chamber and configured to apply a force onto shopping carts in the chamber to cause the shopping carts to move out of the chamber.

3. The system as recited in claim 2, wherein movement of the push bar results in greater movement of the paddle.

4. The system as recited in claim 3, wherein:
the push bar is operatively connected to the paddle by a first set of rollers on a same side of the chamber as the push bar and a second set of rollers on an opposite side of the chamber, and
the second set of rollers exhibit a greater diameter than the first set of rollers.

5. The system as recited in claim 3, wherein the paddle is configured to rotate about an axis in only a single direction.

6. The system as recited in claim 5, wherein the paddle is biased to a neutral position and is configured such that rotation in a first direction from the neutral position is prevented and further such that rotation in a second direction from the neutral position opposite the first direction is permitted.

7. The system as recited in claim 6, wherein the paddle includes a ratchet assembly.

8. The system as recited in claim 6, wherein, when shopping carts are in the chamber, the paddle is permitted to travel from a second end of the chamber to a first end of the chamber without causing movement of the shopping carts and is further permitted to travel from the first end of the chamber to the second end of the chamber while causing movement of the shopping carts.

9. The system as recited in claim 1, wherein the chamber is enclosed by a plurality of panels.

10. The system as recited in claim 9, wherein, within the chamber, the system includes at least one reflective panel.

11. The system as recited in claim 9, wherein at least one of the plurality of panels is a first door arranged at an end of the chamber.

12. The system as recited in claim 11, wherein at least one other of the plurality of panels is a second door arranged at an end of the chamber opposite the first door.

13. The system as recited in claim 1, wherein the at least one UV LED includes a plurality of UV LEDs, and each of the plurality of UV LEDs are configured to emit UV light into the chamber.

14. The system as recited in claim 13, wherein:
the plurality of UV LEDs includes a first row of UV LEDs and a second row of UV LEDs,
the first row of UV LEDs is on a first side of the chamber and is arranged above a height of corresponding to a handlebar of a shopping cart, and
the second row of UV LEDs is on the first side of the chamber and is arranged below the height.

15. The system as recited in claim 14, wherein the first and second rows of UV LEDs are inclined toward the height.

16. The system as recited in claim 14, wherein the first row of UV LEDs includes three light sources and the second row of UV LEDs includes three light sources.

17. The system as recited in claim 14, wherein the plurality of UV LEDs includes an upper row of UV LEDs adjacent a top of the chamber and configured to emit light downward toward the height.

18. The system as recited in claim 1, further comprising a light configured to emit light of a color corresponding to a particular stage of a disinfection cycle.

19. A method, comprising:
disinfecting shopping carts arranged within a chamber by emitting UV light from at least one UV LED within the chamber.

20. The method as recited in claim 19, further comprising:
removing the shopping carts from the chamber after the disinfecting step by applying a force to a push bar, wherein the push bar causes a paddle projecting into the chamber to apply a force onto the shopping carts, and wherein movement of the push bar results in greater movement of the paddle.
